(19) United States
(12) Patent Application Publication
(10) Pub. No.: US 2020/0281619 A1
(43) Pub. Date: Sep. 10, 2020

(54) INSERTABLE ENDOSCOPIC INSTRUMENT FOR TISSUE REMOVAL WITH RETRACTABLE TOOL AT CUTTING TIP

(71) Applicant: Interscope, Inc., Whitinsville, MA (US)

(72) Inventor: Jeffery B. Ryan,, Jr., Whitinsville, MA (US)

(21) Appl. No.: 16/753,179

(22) PCT Filed: Oct. 3, 2018
(86) PCT No.: PCT/US2018/054196

§ 371 Date: Apr. 2, 2020

Related U.S. Application Data
(60) Provisional application No. 62/567,664, filed on Oct. 3, 2017.

(51) Int. Cl. A61B 17/32; A61B 17/00

(52) U.S. Cl. CPC A61B 17/32002; A61B 17/00234; A61B 2217/007; A61B 2017/00269; A61B 2017/00876

(57) ABSTRACT

An endoscopic instrument includes an outer cannula and an inner cannula disposed within the outer cannula, a tool channel, a retractable tool, and a retractable tool actuator. The tool channel is defined within a radial wall of the outer cannula or positioned adjacent to the radial wall of the outer cannula. The retractable tool includes a distal tip and sized to fit within the tool channel The retractable tool actuator is configured to move, responsive to actuation of the retractable tool actuator, the retractable tool along the tool channel from a first position in which the distal tip of the retractable tool is within the tool channel to a second position in which the distal tip of the retractable tool extends beyond a distal end of the outer cannula

### CROSS-REFERENCE TO RELATED APPLICATIONS

The present disclosure claims the benefit of and priority to U.S. Provisional Application No. 62/567,664, titled “INSERTABLE ENDOSCOPIC INSTRUMENT FOR TISSUE REMOVAL WITH RETRACTABLE BLADE AT CUTTING TIP,” filed Oct. 3, 2017, the disclosure of which is incorporated herein by reference in its entirety.

### BACKGROUND

Colon cancer is the third leading cause of cancer in the United States but is the second leading cause of cancer-related deaths. Colon cancer arises from pre-existing colon polyps (adenomas) that occur in as many as 35% of the US population. Colon polyps can either be benign, precancerous or cancerous. Colonoscopy is widely regarded as an excellent screening tool for colon cancer that is increasing in incidence worldwide. According to the literature, a 1% increase in colonoscopy screening results in a 3% decrease in the incidence of colon cancer. The current demand for colonoscopy exceeds the ability of the medical system to provide adequate screening. Despite the increase in colon cancer screening the past few decades, only 55% of the eligible population is screened, falling far short of the recommended 80%, leaving millions of patients at risk.

Due to the lack of adequate resources, operators performing a colonoscopy typically only sample the largest polyps, exposing the patient to sample bias by typically leaving behind smaller less detectable polyps that could advance to colon cancer prior to future colonoscopy. Because of the sample bias, a negative result from the sampled polyps does not ensure the patient is truly cancer-free. Existing polyps removal techniques lack precision are cumbersome and time consuming.

At present, colon polyps are removed using a snare that is introduced into the patient's body via a working channel defined within an endoscope. The tip of the snare is passed around the stalk of the polyp to cut the polyp from the colon wall. Once the cut has been made, the cut polyp lies on the intestinal wall of the patient until it is retrieved by the operator as a sample. To retrieve the sample, the snare is first removed from the endoscope and a biopsy forceps or suction is fed through the same channel of the endoscope to retrieve the sample.

Accordingly, there is a need for an improved endoscopic instrument that increases the precision and speed of polyp removal for biopsy.

### SUMMARY

This Summary is provided to introduce a selection of concepts in a simplified form that are further described below in the Detailed Description. This Summary is not intended to identify key features or essential features of the claimed subject matter, nor is it intended that this Summary be used to limit the scope of the claimed subject matter. Furthermore, the claimed subject matter is not limited to implementations that offer any or all advantages or solve any or all state of the art problems.

An improved endoscopic instrument is provided that can precisely remove sessile polyps and efficiently obtain samples of multiple polyps from a patient. In particular, the improved endoscopic instrument is capable of debriding one or more polyps and retrieving the debrided polyps without having to alternate between using a separate retractable tool and a separate sample retrieving tool. The sampling can be integrated with colonoscopy inspection. In some implementations, the endoscopic instrument can cut and remove tissue from within a patient. In some such implementations, the endoscopic instrument can cut and remove tissue substantially simultaneously from within a patient accessed through a flexible endoscope.

In one aspect, an endoscopic instrument includes an outer cannula and an inner cannula disposed within the outer cannula, a tool channel, a retractable tool, and a retractable tool actuator. The tool channel is defined within a radial wall of the outer cannula or positioned adjacent to the radial wall of the outer cannula. The retractable tool includes a distal tip and sized to fit within the tool channel. The retractable tool actuator is configured to move, responsive to actuation of the retractable tool actuator, the retractable tool along the tool channel from a first position in which the distal tip of the retractable tool is within the tool channel to a second position in which the distal tip of the retractable tool extends beyond a distal end of the outer cannula.

In another aspect, a method of operating an endoscopic instrument includes positioning the endoscopic instrument in proximity to a site of a subject, the endoscopic instrument including an outer cannula and inner cannula disposed within the outer cannula, a tool channel defined within a radial wall of the outer cannula or positioned adjacent to the radial wall of the outer cannula; receiving a control signal at a retractable tool actuator of the endoscopic instrument; moving, by the retractable tool actuator responsive to the control signal, a retractable tool along the tool channel from a first position in which a distal tip of the retractable tool is within the tool channel to a second position in which a distal tip of the retractable tool extends beyond a distal end of the outer cannula; and retrieving a sample of the subject from the site of the subject.

### BRIEF DESCRIPTION OF THE DRAWINGS

The present disclosure is illustratively shown and described in reference to the accompanying drawing in which:

FIG. 1A illustrates various types of polyps that can form within a body.

FIG. 1B is an exploded perspective view of an improved endoscopic instrument according to embodiments of the present disclosure.

FIG. 1C is an end view of the improved endoscopic instrument shown in FIG. 1A according to embodiments of the present disclosure.

FIG. 1D is a cross-sectional view of the improved endoscopic instrument shown in FIG. 1B taken along the section A-A according to embodiments of the present disclosure.

FIG. 2 is a block diagram of an endoscopic instrument including a retractable tool according to embodiments of the present disclosure.

FIG. 3A is a sectional view of a distal end of an endoscopic instrument including a retractable tool operated by a linear actuator in a first configuration according to embodiments of the present disclosure.

FIG. 3B is a sectional view of the endoscopic instrument of FIG. 3A in a second configuration according to embodiments of the present disclosure.

FIG. 3C is a sectional view of a distal end of an endoscopic instrument including a retractable tool operated by a control wire in a first configuration according to embodiments of the present disclosure.

FIG. 3D is a sectional view of a distal end of an endoscopic instrument including a retractable tool operated by electromagnets in a first configuration according to embodiments of the present disclosure.

FIG. 3E is a detail view of the endoscopic instrument of FIG. 3D in the first configuration according to embodiments of the present disclosure.

FIG. 3F is a detail view of the endoscopic instrument of FIG. 3D in a second configuration according to embodiments of the present disclosure.

FIG. 4 is a flow diagram of a method of operating an endoscopic instrument including a retractable tool according to embodiments of the present disclosure.

### DETAILED DESCRIPTION

Technologies provided herein are directed towards an improved flexible endoscopic instrument that can precisely and efficiently obtain samples of single and multiple polyps and neoplasms from a patient. In particular, the improved endoscopic instrument is capable of debriding samples from one or more polyps and retrieving the debrided samples without having to remove the endoscopic instrument from the treatment site within the patient's body.

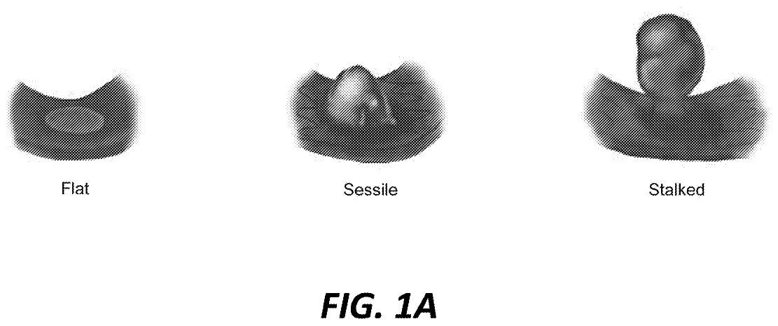

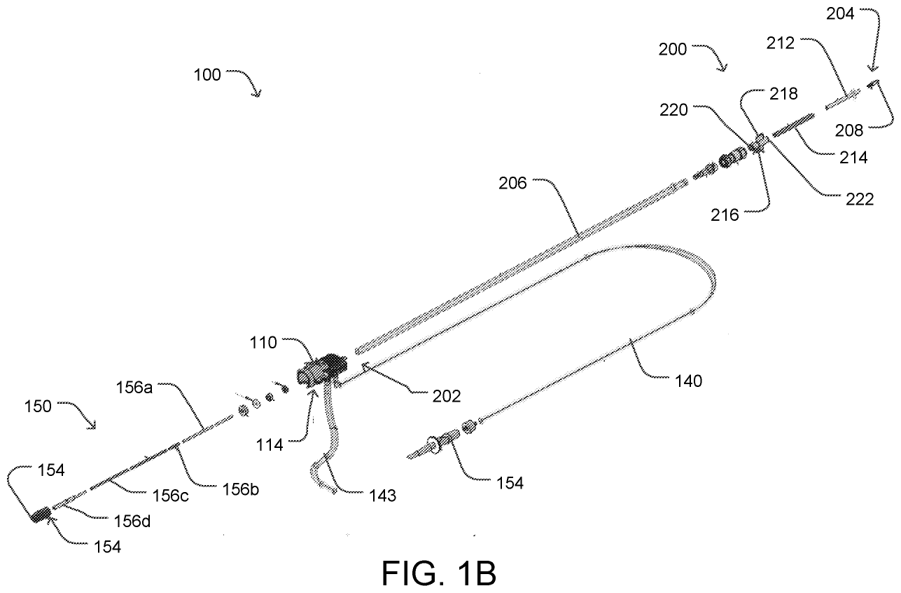

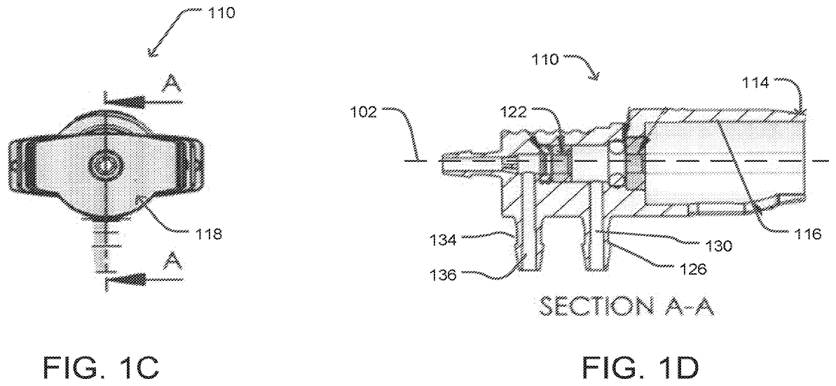

FIG. 1A illustrates various types of polyps that can form within a body. Most polyps may be removed by snare polypectomy, though especially large polyps and/or sessile or flat polyps must be removed piecemeal with biopsy forceps or en bloc using endoscopic mucosal resection (EMR). A recent study has concluded that depressed sessile polyps had the highest rate for harboring a malignancy at 33%. The same study has also found that non-polypoid neoplastic lesions (sessile polyps) accounted for 22% of the patients with polyps or 10% of all patients undergoing colonoscopy. There are multiple roadblocks to resecting colon polyps, namely the difficulties in removing sessile polyps, the time involved in removing multiple polyps and the lack of reimbursement differential for resecting more than one polyp. Since resecting less accessible sessile polyps presents challenges and multiple polyps take more time per patient, most polyps are removed piece meal with tissue left behind as polyps increase in size, contributing to a sampling bias where the pathology of remaining tissue is unknown, leading to an increase in the false negative rate.

Colonoscopy is not a perfect screening tool. With current colonoscopy practices the endoscopist exposes the patient to sample bias through removal of the largest polyps (stalked polyps), leaving behind less detectable and accessible sessile/flat polyps. Sessile polyps are extremely difficult or impossible to remove endoscopically with current techniques and often are left alone. An estimated 28% of stalked polyps and 60% of sessile (flat) polyps are not detected, biopsied or removed under current practice, which contributes to sample bias and a 6% false-negative rate for colonoscopy screening. Current colonoscopy instruments for polyp resection are limited by their inability to adequately remove sessile polyps and inefficiency to completely remove multiple polyps. According to the clinical literature, sessile polyps greater than 10 mm have a greater risk of malignancy. Sessile polyp fragments that are left behind after incomplete resection will grow into new polyps and carry risks for malignancy.

In the recent past, endoscopic mucosal resection (EMR) has been adopted to remove sessile polyps. EMR involves the use of an injection to elevate surrounding mucosa followed by opening of a snare to cut the polyp and lastly use of biopsy forceps or a retrieval device to remove the polyp. The introduction and removal of the injection needle and snare through the length of the colonoscope, which is approximately 5.2 feet, must be repeated for the forceps.

The present disclosure relates to an endoscopic instrument that is capable of delivering an innovative alternative to existing polyp removal tools, including snares, hot biopsy and EMR, by introducing a flexible powered instrument that that works with the current generation colonoscopes and can cut and remove any polyp. The endoscopic instrument described herein can be designed to enable physicians to better address sessile or large polyps as well as remove multiple polyps in significantly less time. Through the adoption of the endoscopic instrument described herein, physicians can become more efficient at early diagnosis of colorectal cancer.

The present disclosure will be more completely understood through the following description, which should be read in conjunction with the drawings. In this description, like numbers refer to similar elements within various embodiments of the present disclosure. Within this description, the claims will be explained with respect to embodiments. The skilled artisan will readily appreciate that the methods, apparatus and systems described herein are merely exemplary and that variations can be made without departing from the spirit and scope of the disclosure.

### A. Endoscopic Instrument

Referring back to the drawings, FIGS. 1B-1D illustrate an endoscopic instrument 100 according to embodiments of the present disclosure. The endoscopic instrument 100 may be similar to various endoscopic instrument described in U.S. patent application Ser. No. 15/459,870, which is incorporated herein by reference in its entirety.

The endoscopic instrument 100 can be configured to obtain samples of polyps and neoplasms from a patient. The endoscopic instrument 100 can be configured to be rotated by a torque source (e.g., a motor coupled to a drive assembly or drive shaft of the endoscopic instrument 100). The endoscopic instrument 100 can be configured to flow irrigation fluid out into a site within a subject (e.g., a site within a colon, esophagus, lung of the subject). The endoscopic instrument 100 can be configured to resect material at a site within a subject. The endoscopic instrument 100 can be configured to provide a suction force via an aspiration channel to obtain a sample of the material resected at a site within a subject. In some implementations, the endoscopic instrument 100 can be configured to be inserted within an instrument channel, such as an instrument channel of an endoscope (e.g., a gastroscope, such as a colonoscope, a laryngoscope, or any other flexible endoscope).

The endoscopic instrument 100 includes a proximal connector 110 and a flexible torque delivery assembly 200. The proximal connector 110 is configured to couple a drive assembly 150 (e.g., a drive assembly including a drive shaft configured to be rotated by a source of rotational energy) of the endoscopic instrument 100 to the flexible torque delivery assembly of the endoscopic instrument 100. In some implementations, the proximal connector 110 includes a first connector end 114 at which the drive assembly 150 is coupled, and a second connector end 118 at which the flexible torque delivery assembly 200 is coupled. As shown in FIGS. 1B and 1D, the first connector end 114 includes an inner wall 116 defining an opening in which the drive assembly 150 can be received. For example, in some implementations, the proximal connector 110 can be used to connect the drive assembly 150 to a drive shaft of a surgical console. The proximal connector 110 includes a drive transfer assembly 122. The drive transfer assembly 122 is configured to be operatively coupled to the drive assembly 150, receive torque from the drive assembly 150 when the drive assembly 150 rotates, and transfer the torque to the flexible torque delivery assembly 200 in order to rotate the flexible torque delivery assembly 200. In some implementations, the drive assembly 150, drive transfer assembly 122, and at least a portion of the flexible torque delivery assembly 200 are coaxial. For example, the drive transfer assembly 122 can be engaged to the drive assembly 150 along a drive axis 102, and the drive transfer assembly 122 can also be engaged to the flexible torque delivery assembly 200 at a proximal end 204 of the flexible torque delivery assembly 200 along the drive axis 102. It should be appreciated that rotating the flexible torque delivery assembly may include causing the flexible torque delivery assembly to rotate a component (such as an inner cannula) at one of the flexible torque delivery assembly.

In some implementations, the drive transfer assembly 122 includes gears, belts, or other drive components to control the direction and/or torque transferred from the drive assembly 150 to the flexible torque delivery assembly 200. For example, such drive components can be positioned at an angle to one another to change an axis of rotation of the flexible torque delivery assembly 200, or offset from one another to shift an axis of rotation of the flexible torque delivery assembly 200 relative to the drive axis 102.

In some implementations, the drive assembly 150 includes a drive engagement member 152. The drive engagement member 152 is configured to engage the drive assembly 150 to a source of rotational energy (e.g., a drive rotated by a motor, such as console drive assembly of a surgical console). The drive engagement member 152 can be configured to be fixedly and/or rigidly connected to the console drive assembly, such that the drive engagement member 152 rotates in unison with the console drive assembly. For example, as shown in FIG. 1B, the drive engagement member 152 includes a proximal drive end 154 including a fitting (e.g., hex fitting, pin fitting, etc.) configured to engage (e.g., lock with, mate with, fixedly engage, frictionally engage, etc.) a console drive assembly. As such, rotation of the console drive assembly causes rotation of the drive engagement member 152.

In some implementations, the drive assembly 150 includes one or more shaft components 154 configured to transfer rotation of the drive engagement member 150 to the drive transfer assembly 122. In some implementations, the drive transfer assembly 122 includes the one or more shaft components 156. The shaft components 156 can include an insulator member 156a (e.g., a heat sheath, heat shrink, etc.) configured to insulate components of the drive assembly 150 from heat generated by rotation of the drive assembly or components thereof. The shaft components 156 can include a cutter 156b. The shaft components 156 can include a shaft torque coil 156c which may be similar to other torque coils described herein. In some implementations, the shaft components 156 can include a shaft torque rope. The shaft components 156 can include a shaft tube 156d. The shaft tube 156d can include a radius that is less than a relatively greater radius of the drive engagement member 152 (e.g., a relatively greater radius that may facilitate receiving rotational energy from a drive shaft or other rotational energy source, such as by engaging the drive engagement member 152 to a console drive assembly). For example, the shaft tube 156d can include a relatively lesser smaller corresponding more closely to a radius of the drive transfer assembly 122 and/or the flexible torque delivery assembly 200. In such implementations, the torque received at the drive transfer assembly 122 and/or the flexible torque delivery assembly 200 can be modified (e.g., increased) in a manner corresponding to the change in radius between the radius of the drive engagement member 152 and the radius of the shaft tube 156d.

In some implementations, the cutting assembly 201 can include an outer cannula and an inner cannula disposed within the outer cannula. The outer cannula can define an opening 208 through which material to be resected can enter the cutting assembly 201. In some implementations, the opening 208 is defined through a portion of the radial wall of the outer cannula. In some implementations, the opening 208 may extend around only a portion of the radius of the outer cannula, for example, up to one third of the circumference of the radial wall. As the aspiration channel extends between a vacuum port (e.g., vacuum port 126) and the opening 208, any suction applied at the vacuum port causes a suction force to be exerted at the opening 208. The suction force causes material to be introduced into the opening or cutting window of the outer cannula, which can then be cut by the inner cannula of the cutting assembly 201.

The inner cannula can include a cutting section that is configured to be positioned adjacent to the opening 208 such that material to be resected that enters the cutting assembly 201 via the opening 208 can be resected by the cutting section of the inner cannula. The inner cannula may be hollow and an inner wall of the inner cannula may define a portion of an aspiration channel that may extend through the length of the endoscopic instrument. A distal end of the inner cannula can include the cutting section while a proximal end of the inner cannula can be open such that material entering the distal end of the inner cannula via the cutting section can pass through the proximal end of the inner cannula. In some implementations, the distal end of the inner cannula can come into contact with an inner surface of a distal end of the outer cannula. In some implementations, this can allow the inner cannula to rotate relative to the outer cannula along a generally longitudinal axis, providing more stability to the inner cannula while the inner cannula is rotating. In some implementations, the size of the opening can dictate the size of the materials being cut or resected by the inner cannula. As such, the size of the opening may be determined based in part on the size of the aspiration channel defined by the inner circumference of the flexible torque coil.

The endoscopic instrument 100 can include a flexible torque coil 212 that is configured to couple to the proximal end of the inner cannula at a distal end of the flexible torque coil 212. The flexible torque coil can include a fine coil with multiple threads and multiple layers, which can transmit the rotation of one end of the flexible torque coil to an opposite end of the flexible torque coil. Each of the layers of thread of the flexible torque coil can be wound in a direction opposite to a direction in which each of the layers of thread adjacent to the layer of thread is wound. In some implementations, the flexible torque coil can include a first layer of thread wound in a clockwise direction, a second layer of thread wound in a counter-clockwise direction and a third layer of thread wound in a clockwise direction. In some implementations, the first layer of thread is separated from the third layer of thread by the second layer of thread. In some implementations, each of the layers of thread can include one or more threads. In some implementations, the layers of thread can be made from different materials or have different characteristics, such as thickness, length, among others.

The flexibility of the torque coil 212 allows the coil to maintain performance even in sections of the torque coil 212 that are bent. Examples of the flexible torque coil 212 include torque coils made by ASAHI INTECC USA, INC located in Santa Ana, Calif., USA. In some implementations, the flexible torque coil 212 can be surrounded by a sheath or lining (e.g., sheath 214) to avoid frictional contact between the outer surface of the flexible torque coil 212 and other surfaces. In some implementations, the flexible torque coil 212 can be coated with Polytetrafluoroethylene (PFTE) to reduce frictional contact between the outer surface of the flexible torque coil 212 and other surfaces. The flexible torque coil 212 can be sized, shaped or configured to have an outer diameter that is smaller than the diameter of the instrument channel of the endoscope in which the endoscopic instrument is to be inserted. For example, in some implementations, the outer diameter of the flexible torque coil can be within the range of 1-4 millimeters. The length of the flexible torque coil can be sized to exceed the length of the endoscope. In some implementations, the inner wall of the flexible torque coil 212 can be configured to define another portion of the aspiration channel that is fluidly coupled to the portion of the aspiration channel defined by the inner wall of the inner cannula of the cutting assembly 201. A proximal end of the flexible torque coil 212 can be coupled to the proximal connector 110 (e.g., to the drive transfer assembly 122 of the proximal connector 110, etc.).

The endoscopic instrument 100 can include a flexible outer tubing 206 that can be coupled to the proximal end of the outer cannula. In some implementations, a distal end of the flexible outer tubing 206 can be coupled to the proximal end of the outer cannula using a coupling component. In some implementations, the outer cannula can be configured to rotate responsive to rotating the flexible outer tubing. In some implementations, the flexible outer tubing 206 can be a hollow, braided tubing that has an outer diameter that is smaller than the instrument channel of the endoscope in which the endoscopic instrument 100 is to be inserted. In some implementations, the length of the flexible outer tubing 206 can be sized to exceed the length of the endoscope. The flexible outer tubing 206 can define a bore through which a portion of the flexible outer tubing 206 extends. The flexible outer tubing 206 can include braids, threads, or other features that facilitate the rotation of the flexible outer tubing 206 relative to the flexible torque coil, which is partially disposed within the flexible outer tubing 206. The flexible outer tubing can define a portion of an irrigation channel for outputting fluid to a site within a subject.

The endoscopic instrument 100 can include a rotational coupler 216 configured to be coupled to a proximal end of the flexible outer tubing 206. The rotational coupler 216 may be configured to allow an operator of the endoscopic instrument to rotate the flexible outer tubing 206 via a rotational tab 218 coupled to or being an integral part of the rotational coupler 216. By rotating the rotational tab 218, the operator can rotate the flexible outer tubing and the outer cannula along a longitudinal axis of the endoscope and relative to the endoscope and the inner cannula of the cutting assembly 201. In some implementations, the operator may want to rotate the outer cannula while the endoscopic instrument is inserted within the endoscope while the endoscope is within the patient. The operator may desire to rotate the outer cannula to position the opening of the outer cannula to a position where the portion of the radial wall of the outer cannula within which the opening is defined may aligned with the camera of the endoscope such that the operator can view the material entering the endoscopic instrument for resection via the opening. This is possible in part because the opening is defined along a radial wall extending on a side of the outer cannula as opposed to an opening formed on the axial wall of the outer cannula.

In some implementations, a proximal end 220 of the rotational coupler 216 can be fluidly coupled to the proximal connector 110, such that the irrigation channel of the endoscopic instrument 100 passes from an irrigation port 134 through the flexible outer tubing 206 into the rotational coupler 216. Irrigation fluid entering the proximal connector 110 at the irrigation port 134 can thus pass through the rotational coupler 216 in order to be outputted at a site within a subject. In some implementations, the rotational coupler 216 can be a rotating luer component that allows a distal end 222 of the rotational coupler 216 to rotate relative to the proximal end 220 of the rotational coupler 216. In this way, when the flexible outer tubing 206 is rotated, the component to which the proximal end of the rotational coupler 216 is coupled, is not caused to rotate. The rotational coupler 216 can define a bore along a central portion of the rotational coupler 216 through which a portion of the flexible torque coil 212 extends. In some implementations, the rotational coupler 216 can be a male to male rotating luer connector. In some implementations, the rotational coupler can be configured to handle pressures up to 1200 psi.

In some implementations, the flexible torque delivery assembly 200 is configured to be fluidly coupled to a vacuum source to apply a suction force to the aspiration channel. The aspiration channel allows for fluid and material (e.g., a sample to be obtained) to be drawn into the distal end 204 of the flexible torque delivery assembly 200 in order to flow to the proximal end 202 of the flexible torque delivery assembly 200. For example, after the cutting assembly 201 has been used to resect material from a site within a subject, vacuum pressure can be applied through the aspiration channel to draw (e.g., transfer by suction, etc.) fluid and material into the flexible torque delivery assembly 200.

In some implementations, the proximal connector 110 is configured to be coupled to a vacuum source to provide a suction force for aspiration. For example, as shown in FIGS. 1B and 1D, the proximal connector 110 includes a vacuum port 126 (e.g., aspiration port). The vacuum port/aspiration port 126 can be similar to other aspiration ports disclosed herein. The vacuum port 126 is configured to fluidly couple an aspiration channel of the endoscopic instrument 100 to a vacuum source (e.g., to a vacuum source with a specimen receiver positioned between the vacuum source and the endoscopic instrument). The vacuum port 126 is configured to transmit a suction force applied to the vacuum port 126 to the aspiration channel, in order to draw fluid and material entering the distal end 204 of the endoscopic instrument 100 through the aspiration channel towards the vacuum source. In some implementations, such as shown in FIGS. 1B and 1D, the vacuum port 126 includes a vacuum port channel 130 oriented transverse to the drive axis 102 (and thus the aspiration channel). This may facilitate coupling tubing to the vacuum port 126 that extends to a specimen receiver or vacuum source without interfering with manipulation of the proximal connector 110 and the endoscopic instrument 100. In various implementations, the vacuum port channel 130 can be oriented at varying angles relative to the drive axis 102. In some implementations, vacuum tubing 132 can be coupled to the vacuum port 126.

In some implementations, the proximal connector 110 is configured to be coupled to a fluid source to provide fluid to be outputted by the endoscopic instrument 100 to a site within a subject. As shown in FIGS. 1B-1D, the proximal connector 110 includes an irrigation port 134, including an irrigation port channel 136, configured to receive fluid from a fluid source. The irrigation port 134 is configured to be fluidly coupled to an irrigation channel of the flexible torque delivery assembly 200 (e.g., an irrigation channel defined between the flexible outer tubing 206 and the flexible torque coil 212 and extending to an opening at the distal end 204 of the flexible torque delivery assembly 200), such that fluid can flow from the proximal connector 110 through flexible torque delivery assembly 200 to be outputted at a site within a subject. In some implementations, the fluid (e.g., irrigation fluid) can be used to cool the flexible torque delivery assembly 200, which may generate heat due to friction caused by rotation or other movements. In some implementations, the fluid can be used to wash a site within a subject. In some implementations, the fluid provides lubrication to facilitate rotation or other movement of components of the endoscopic instrument 100 relative to one another. In some implementations, the irrigation port 134 is configured to be coupled to a fluid transfer device or irrigation pump. The irrigation port 134 receives a flow of irrigation fluid from the irrigation pump and transfers the fluid into the irrigation channel. In some implementations, the irrigation channel is defined to include the irrigation port 134 and/or tubing connecting the irrigation port 134 to the fluid source. In some implementations, the irrigation port 134 can be coupled to a fluid source by fluid tubing 140. The fluid tubing 140 can be coupled to a fitting 144 (e.g., vented spike fitting, non-vented spike fitting, etc.) configured to interface the fluid tubing 140 to a fluid source.

### B. Systems and Methods for Insertable Endoscopic Instrument for Tissue Removal with Retractable Tool at Cutting Tip

In existing endoscopic instrument systems, a cutting assembly may be provided which can be rotated to resect polyps and other materials from a site within a subject. However, in certain use cases or procedures, the cutting assembly may not be able to effectively resect desired material, such as to resect relatively large portions of polyps adjacent to where the polyps protrude from underlying tissue.

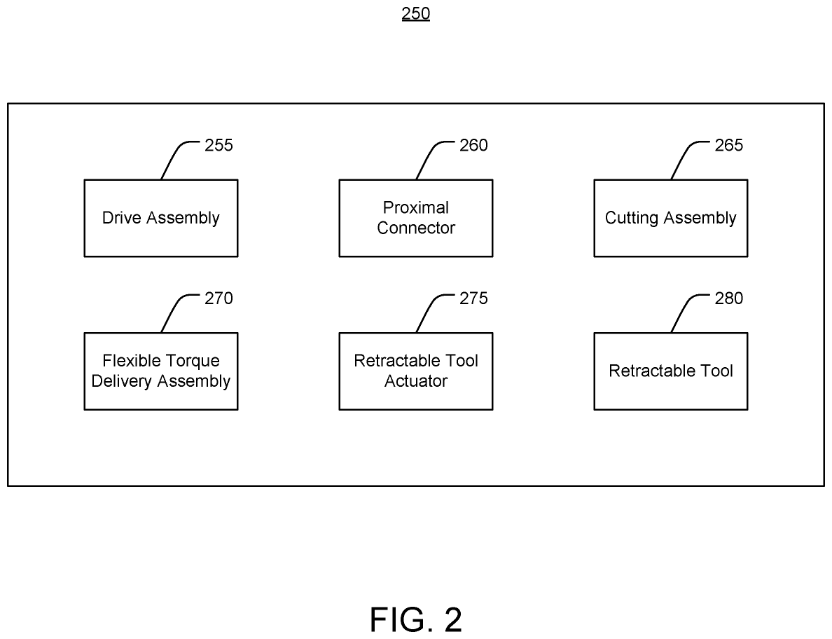

FIG. 2 illustrates a block diagram of an endoscopic instrument 250 including a retractable blade according to embodiments of the present disclosure. The endoscopic instrument 250 may incorporate features of the endoscopic instrument 100 described with reference to FIGS. 1B-1D. In some implementations, the endoscopic instrument 250 includes a drive assembly 255, a proximal connector 260, a cutting assembly 265, and a flexible torque delivery assembly 270. The drive assembly 255 can be coupled to the flexible torque delivery assembly 270 via the proximal connector 260 to cause the flexible torque delivery assembly 270 to rotate.

In some implementations, the endoscopic instrument 200 includes a retractable tool 275 and a retractable tool actuator 280. The retractable tool 275 is configured to resect material at a site within a subject. The retractable tool 275 can be configured to move in a direction transverse to a direction in which the cutting assembly 265 rotates, which may enable greater the endoscopic instrument 250 to be used for a greater range of procedures and tissue manipulation while maintaining a compact form factor useful for endoscopic procedures.

The retractable tool 275 may be disposed at a distal end of the endoscopic instrument 250 in a manner similar to an instrument channel, camera, or camera lens of various endoscopic instruments described herein. In some embodiments, the retractable tool 275 is disposed closer to an outer surface of the endoscopic instrument 250 than a longitudinal axis of the endoscopic instrument 250. As such, rotation of the endoscopic instrument 250 about the longitudinal axis of the endoscopic instrument 250 may allow the retractable tool 275 to reach various locations around the site within the subject which would otherwise be inaccessible to cutting assembly 265 (e.g., if the cutting assembly 265 is located along the longitudinal axis).

The retractable tool 275 can be configured to cut, resect, excise, or otherwise remove a sample of material (e.g., tissue) at the site in the subject. The retractable tool 275 may include a relatively thin edge extending in a direction generally parallel to the longitudinal axis of the endoscopic instrument 250. In some embodiments, the edge of the retractable tool 275 is serrated. The retractable tool 275 may be made from a material such as stainless steel or titanium. The retractable tool 275 may be made from a biocompatible material. The retractable tool 275 may have a rigidity greater than a threshold rigidity sufficient to resect the sample of material, given a surface area to volume ratio of the retractable tool 275. In some implementations, the retractable tool 275 includes one or more blades.

The retractable tool 275 can be configured to be manipulated (e.g., moved relative to the endoscopic instrument 250, such as by being moved out of or into the endoscopic instrument 250) by the retractable tool actuator 280. In some implementations, the retractable tool actuator 280 includes a linear actuator. The retractable tool actuator 280 can be configured to drive the retractable tool 275 from a first position (e.g., a retracted position) to a second position (e.g., an extended position) and back to the first position. At the first position, a distal end of the retractable tool 275 may be disposed within the endoscopic instrument 250. At the second position, the distal end of the retractable tool 275 may extend out of the endoscopic instrument 250.

The benefit of having a retractable retractable tool 275 is to reduce the risk of injury to the subject while the retractable tool is not in use or operation. As a surgeon manipulates the endoscopic instrument 250 within the subject, the retractable tool 275 can be maintained in the retracted position such that the retractable tool 275 is not able to contact any organs, such as the colon, esophagus or other part of the subject while the endoscopic instrument is inserted within an endoscope that is inserted within the subject. At a time when the surgeon desires to use the retractable tool 275, the surgeon may deploy the retractable tool from the retracted position to the extended position for use. After the surgeon no longer needs the retractable tool 275, the surgeon may retract the retractable tool 275 from the deployed position to the retracted position. Both the deployment and retraction of the retractable tool 275 from the endoscopic instrument 250 can be done without having to remove the endoscopic instrument from within the subject or the endoscope within which it is inserted.

It should be appreciated that the retractable tool 275 and the deployment and retraction mechanisms described herein can be implemented in any medical device where there is a need to retract or stow away the retractable tool while the retractable tool is not in use.

In some implementations, the retractable tool actuator 280 is configured to control operation of the retractable tool 275 based on a control signal. The retractable tool actuator 280 can be configured to receive the control signal via a control line (not shown) extending within the endoscopic instrument 250 from the proximal end of the endoscopic instrument 250 to the retractable tool actuator 280. The retractable tool actuator 280 can be configured to execute control of the retractable tool 275 based on a voltage magnitude, pulse width, or other parameter of the control signal. In some implementations, the retractable tool actuator 280 includes a processing circuit configured to receive the control signal and control operation of the retractable tool 275 based on the control signal. The retractable tool actuator 280 can be configured to control at least one of a distance the retractable tool 275 extends out of the endoscopic instrument 250 or a frequency of movement of the retractable tool 275 (e.g., based on the control signal).

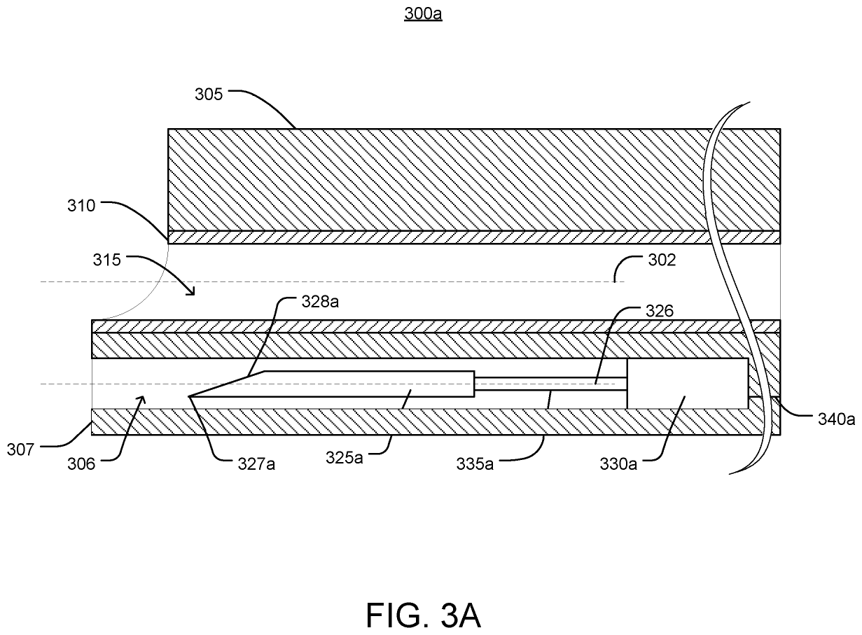

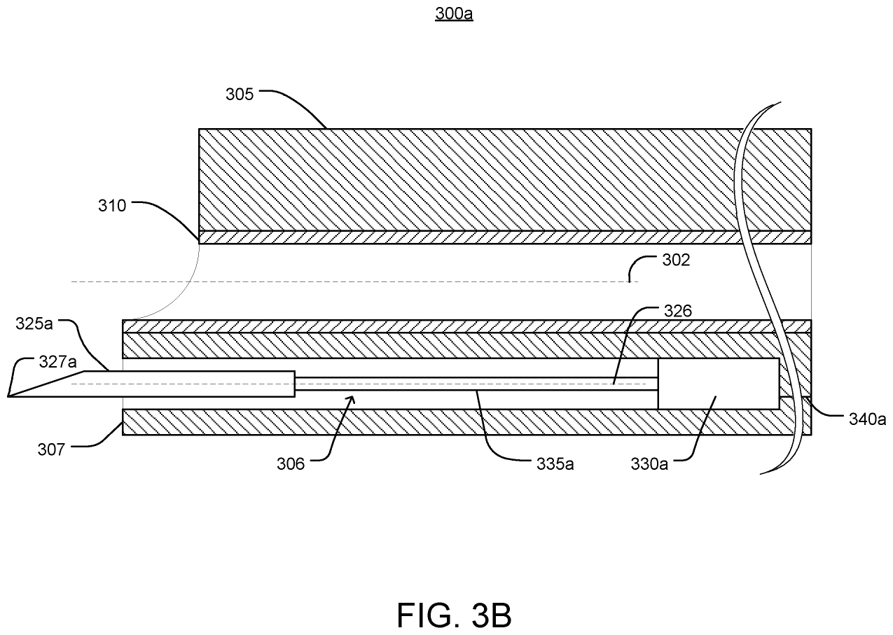

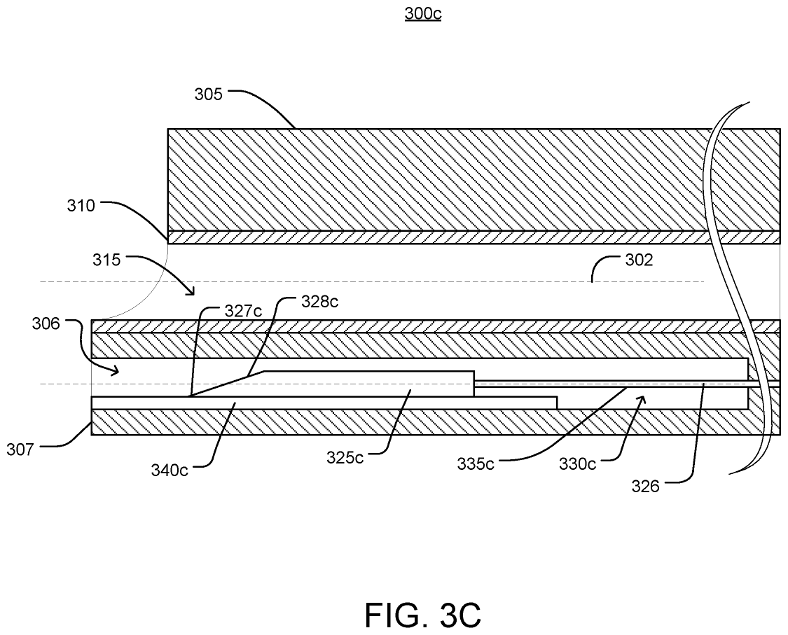

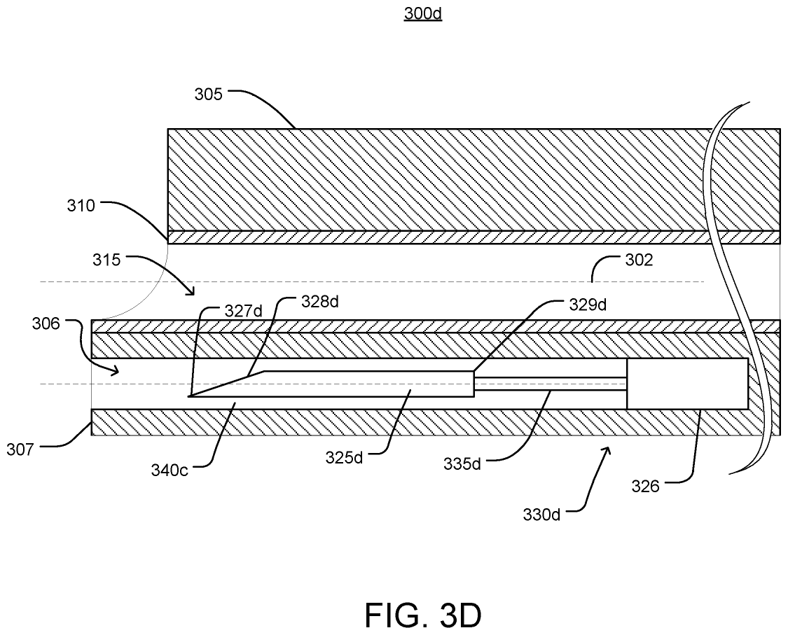

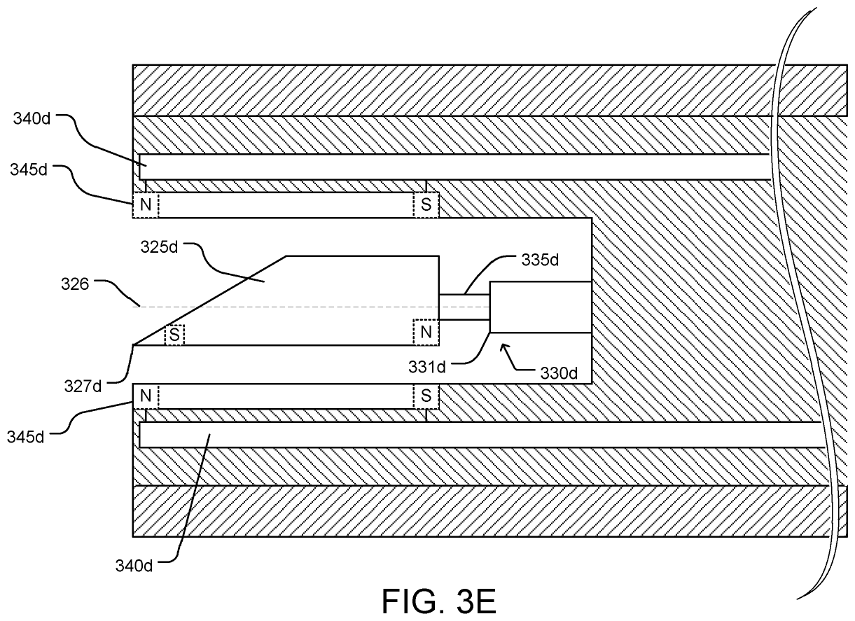

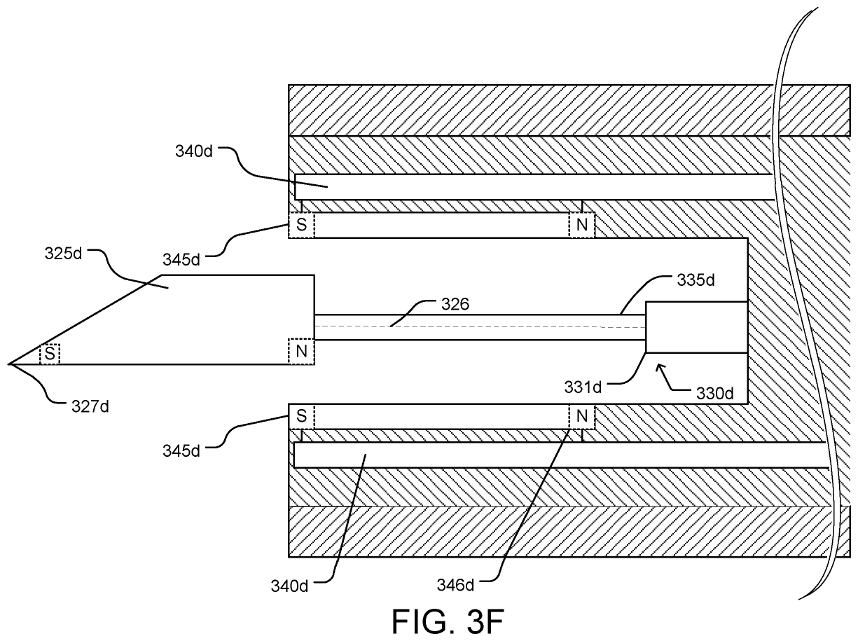

Referring now to FIGS. 3A-3B, an endoscopic instrument 300a is illustrated according to embodiments of the present disclosure. The endoscopic instrument 300a can incorporate features of the endoscopic instrument 200 described with reference to FIG. 2. In some implementations, the endoscopic instrument 300a includes an outer cannula 305 and an inner cutter 310 defining an inner cannula 315. The inner cutter 310 can be configured to be rotated (e.g., by flexible torque delivery assembly 215) about a longitudinal axis 302 of the endoscopic instrument 300a, such as to resect material contacted by the inner cutter 310. The material may be drawn into the inner cannula 315 (e.g., via a vacuum force applied through an aspiration channel).

The endoscopic instrument can define a tool channel 306, which may be defined within a radial wall of the outer cannula 305 or positioned adjacent to the radial wall of the outer cannula 305. The endoscopic instrument 300a includes a retractable tool 325a and a retractable tool actuator 330a, which may be disposed in the tool channel 306. As shown in FIG. 3A, the retractable tool 325a can be connected to the retractable tool actuator 330a by a shaft 335a. For example, the retractable tool actuator 330a can be configured to drive the shaft 335a along a drive axis 326 to move the retractable tool 325a out of (or back into) the tool channel 306. In some implementations, the retractable tool actuator 330a is configured to move the retractable tool 325a from a first position (e.g., as shown in FIG. 3A) to a second position (e.g., as shown in FIG. 3B). For example, in the first position, a distal end 327a of the retractable tool 325a may be disposed within the tool channel 306 (e.g., the distal end 327a is inward of distal edge 307 of the outer cannula 305). In the second position, the distal end 327a may be disposed outside the tool channel 306.

The retractable tool 325a includes a cutting edge 328a. As shown in FIG. 3A, the cutting edge 328a extends from the distal end 327a (e.g., on a side of the retractable tool 325a distal to the longitudinal axis 302) towards the longitudinal axis 302 and the retractable tool actuator 330a (e.g., towards a proximal end of the endoscopic instrument 300a). The cutting edge 328a can be configured to resect material at the site within the subject, such as by being moved back and forth along a boundary of the material to be resected. The cutting edge 328a may include a serrated surface.

The retractable tool actuator 330a can be configured to control operation of the retractable tool 325a based on a control signal received via a control line 340a. The control line 340a can be configured to receive the control signal from a user interface (not shown); for example, the user interface can be configured to receive a user input and generate the control signal based on the user input. The retractable tool actuator 330a can be configured to determine a control parameter for controlling operation of the retractable tool 325a, based on the control signal. The control parameter may include one or more of a movement duration, movement frequency, or movement intermittency for movement of the retractable tool 325a. The retractable tool actuator 330a can be configured to receive electrical power via the control line 340a or a separate power line (not shown). The retractable tool actuator 330a may include a motor configured to be driven by electrical power, or a piezoelectric element configured to oscillate in response to receiving electrical power.

In some implementations, the retractable tool actuator 330a receives electrical power as an electrical signal from the control line 340a, where the electrical signal also carries the control signal. For example, the electrical signal received from the control line 340a can be modulated (e.g., modulated in voltage) in accordance with the control signal, such that an electric motor, piezoelectric element, or other drive element of the retractable tool actuator 330a can be activated based on power delivered by the modulated electrical signal.

In some implementations, the retractable tool actuator 330a includes a linear actuator configured to drive the retractable tool 325a (e.g., by shaft 335a) along the tool axis 326. The linear actuator can include a motor configured to generate rotational motion, and a drive shaft connected to the motor to convert the rotational motion to reciprocal motion; the drive shaft may include or be coupled to the shaft 335a to cause linear motion of the retractable tool 325a. In some implementations, the retractable tool actuator 330a includes a linear encoder configured to output a signal indicating a position of the shaft, which may correspond to the position of the retractable tool 325a.

The retractable tool actuator 330a can be configured to move the retractable tool 325a at the movement frequency, which may correspond to a rate at which the retractable tool 325a moves along the tool axis 326 (e.g., a rate at which the distal end 327a moves past a reference point, such as a point where the tool axis 326 intersects a plane in which the distal edge 307 lies). Similarly, the retractable tool actuator 330a can be configured to move the retractable tool 325a based on the movement duration and/or movement intermittency. In some implementations, the retractable tool actuator 330a is configured to deliver electricity into the retractable tool 325a, which may enable the retractable tool 325a to perform electrocautery.

The retractable tool actuator 330a may be attached to the inner cutter 315 or the outer cannula 305. For example, the retractable tool actuator 330a can be configured to be rotated together with the inner cutter 315 or the outer cannula 305 while attached to the respective component.

Referring now to FIG. 3C, an endoscopic instrument 300c is illustrated according to embodiments of the present disclosure. The endoscopic instrument 300c can be similar to the endoscopic instrument 300a, with the exception of the operation of the retractable tool actuator 330c as described below. The endoscopic instrument 300c can include a retractable tool 325c including a distal end 327c and a cutting edge 328c, and a retractable tool actuator 330c.

In some implementations, the retractable tool actuator 330c includes a control wire 335c. The control wire can extend from a proximal end of the endoscopic instrument 300 (e.g., adjacent to a proximal connector such as the proximal connector 205 described with reference) to the retractable tool 325c disposed in the tool channel 306.

The control wire 335c can include or be connected to a biasing element (e.g., a spring) disposed near the distal end of the endoscopic instrument 300c. The biasing element can be configured to bias the retractable tool 325c to the first position (e.g., the position shown in FIG. 3C), such that a force applied to the retractable tool 325c via the control wire may be greater than a bias force of the biasing element to move the retractable tool 325c out of the tool channel 306. The control wire may include or be coupled to a pulley system or other mechanism configured to convert a force applied in a direction away from the distal end of the endoscopic instrument 300c into a force applied in a direction towards the distal end of the endoscopic instrument 300c, which may enable an operator of the endoscopic instrument 300c to apply a force to the control wire 335c (e.g., pull a proximal portion of the control wire 335c away from the distal end of the endoscopic instrument 300c) to cause the retractable tool 325c to move out of the tool channel 306; when the control wire 335c is not receiving the force, the biasing element may return the retractable tool 325c to the first position. The retractable tool 325c may be configured to move to a second position (e.g., a position similar to the second position shown in FIG. 3B for endoscopic instrument 300a) in response to receiving a force from the control wire 335c.

The endoscopic instrument 300c can include one or more track elements 340c. The track element 340c can include a slot configured to receive the retractable tool 325c, which may stabilize the retractable tool 325c as the retractable tool 325c moves in or out of the tool channel 306.

Referring now to FIGS. 3D-3F, an endoscopic instrument 300d is illustrated according to embodiments of the present disclosure. The endoscopic instrument 300d can be similar to the endoscopic instruments 300a, 300c, with the exception of the operation of the retractable tool 325d and retractable tool actuator 330d as described below. The endoscopic instrument 300d can include a retractable tool 325d including a distal end 327d and a cutting edge 328d, and a retractable tool actuator 330d.

In some implementations, the retractable tool 325d includes a permanent magnet. For example, the retractable tool 325d may be made from a ferromagnetic material. As shown in FIGS. 3E-3F, the retractable tool 325d may have a first magnetic pole (e.g., north pole) at the distal end 327d, and a second magnetic pole (e.g., south pole) at a proximal end 329d opposite the distal end. It will be appreciated that the polarity of the retractable tool 325d may be reversed.

The retractable tool actuator 330d can include a shaft 335d along which the retractable tool 325d can translate. For example, the retractable tool 325d can translate from a first position (e.g., as shown in FIG. 3E) to a second position (e.g., as shown in FIG. 3F). In some implementations, the retractable tool actuator 330d includes a stop 331d configured to limit translation of the retractable tool 325d in a direction towards the proximal end of the endoscopic instrument 300d.

In some implementations, the endoscopic instrument 300d includes one or more electric power lines 340d. The electric power lines 340d are configured to carry electrical power from a proximal end of the endoscopic instrument 300d to the distal end shown in FIGS. 3D-3F. Each electric power line 340d may include one or more electrical wires configured to deliver electrical power. The endoscopic instrument 300d also includes one or more electromagnets 345d, which can receive electricity from the electric power lines 340d and generate a magnetic field having a first magnetic pole and a second magnetic pole. The magnitude of the magnetic field may be controlled based on a magnitude of electric current delivered to the electromagnet 345d via the electric power lines 340d. In the configuration illustrated in FIG. 3E, the north pole of the electromagnet 345d is adjacent to the distal end of the endoscopic instrument 300d, while the south pole of the electromagnet 345d is away from the distal end of the endoscopic instrument 300d.

Using the magnetic field generated by the electromagnet 345d, the endoscopic instrument 300d can be configured to hold the retractable tool 325d in one or more stable positions. For example, at the one or more stable positions, a force balance on the retractable tool 325d include magnetic forces from the electromagnet 345d is zero. It will be appreciated that the electromagnet 345d and retractable tool 325d can be configured so that the magnetic force generated by the electromagnet 345d is sufficiently large compared to other forces which may be applied to the retractable tool 325d (e.g., gravity, pressure from fluid near the site of the subject) that such other forces may be negligible in controlling operation of the retractable tool 325d. As shown in FIG. 3E, the first position may be a stable position, where south pole of the retractable tool 325d is closest to the distal, north poles of the electromagnets 345d, and the north pole of the retractable tool 325d is closest to the proximal, south poles of the electromagnets 345d.

As shown in FIG. 3F, the polarities of the electromagnets 345d have been reversed as compared to the configuration of FIG. 3E. As such, a resultant force may be applied to the retractable tool 325d (e.g., due to magnetic poles of like polarity being adjacent to one another), causing the retractable tool 325d to be driven out of the tool channel 306 to the second position shown in FIG. 3F. The second position may also be a stable position; in some implementations, a distal end of the shaft 335d may include one or more stops (not shown) configured to limit translation of the retractable tool 325d in a direction away from the proximal end of the endoscopic instrument 300d.

In various implementations, the magnitude of the electric current delivered to different electromagnets 345d may be different, which may allow differing control schemes for movement of the retractable tool 325d. In various implementations, the endoscopic instrument 300d may include a plurality of electromagnets 345d each configured to individually receive electrical power form the electric power lines 340d, which can enable the electromagnets 345d to be turned on or off individually, such as for allowing sequential activation of the electromagnets 345d as the retractable tool 325d moves along the cutting axis 326.

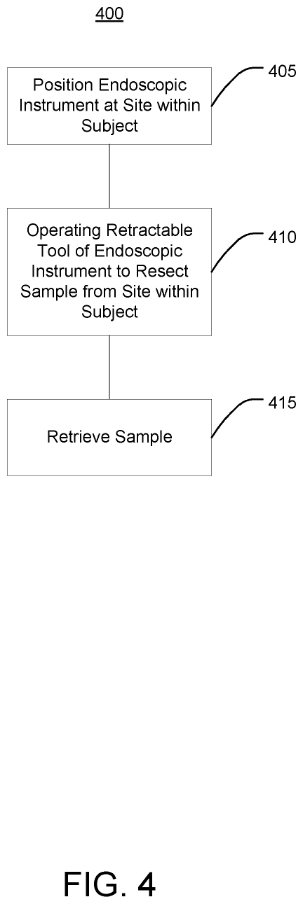

Referring now to FIG. 4, a flow diagram of a method 400 of operating an endoscopic instrument including a retractable blade is shown according to embodiments of the present disclosure. The method may be performed using various endoscopic instruments described herein (e.g., endoscopic instruments 200, 300a, 300c, 300d). The method may be performed using a surgical console or other user interface configured to control operation of the endoscopic instrument. The method may be performed by a surgeon, technician, or other medical professional.

At 405, an endoscopic instrument is positioned in proximity to a site of a subject. The site of the subject may include a sample desired to be resected, such as a polyp within a colon of the subject, or other tissue to be resected. Positioning of the endoscopic instrument may be monitored using a camera of the endoscopic instrument. The endoscopic instrument may include an outer cannula and inner cannula disposed within the outer cannula. A tool channel may be defined within a radial wall of the outer cannula or positioned adjacent to the radial wall of the outer cannula

At 410, a retractable tool actuator of the endoscopic instrument is operated. The retractable tool actuator may be attached to a retractable tool (e.g., blade) to cause the retractable tool to move along a tool axis. The cutting tool may extend in a direction parallel to a longitudinal axis of the endoscopic instrument. The retractable tool actuator may include a linear actuator. In some implementations, operating the retractable tool actuator includes receiving a control signal at the retractable tool actuator to cause the retractable tool actuator to move the retractable tool. The retractable tool actuator may control operation of the retractable tool based on the control signal. Operating the retractable tool may include moving the retractable tool in and out of a tool channel of the outer cannula along a surface of the sample desired to be resected.

At 415, the sample may be retrieved. Retrieving the sample may include positioning an aspiration channel of the endoscopic instrument (e.g., an aspiration channel fluidly coupled to the inner cannula) adjacent to the sample to apply a vacuum force on the sample and pull the sample from the distal end of the endoscopic instrument to a proximal end of the endoscopic instrument.

What is claimed is:

1. An endoscopic instrument, comprising:
an outer cannula and an inner cannula disposed within the outer cannula;
a tool channel defined within a radial wall of the outer cannula or positioned adjacent to the radial wall of the outer cannula;
a retractable tool including a distal tip and sized to fit within the tool channel;
a retractable tool actuator configured to move, responsive to actuation of the retractable tool actuator, the retractable tool along the tool channel from a first position in which the distal tip of the retractable tool is within the tool channel to a second position in which the distal tip of the retractable tool extends beyond a distal end of the outer cannula.

2. The endoscopic instrument of claim 1, comprising:
an aspiration channel extending from an opening of the inner cannula at which material is removed from a site within a subject by the inner cannula to a proximal end of the endoscopic instrument.

3. The endoscopic instrument of claim 1, comprising:
an endoscope defining an instrument channel, the endoscopic instrument extending through the instrument channel.

4. The endoscopic instrument of claim 1, wherein the retractable tool is made from at least one of stainless steel, titanium, or a biocompatible material.

5. The endoscopic instrument of claim 1, wherein the retractable tool actuator includes a linear actuator to move the retractable tool from the first position to the second position.

6. The endoscopic instrument of claim 1, wherein the retractable tool actuator controls at least one of a distance the retractable tool extends out of the tool channel or a frequency of movement of the retractable tool based on a received control signal.

7. The endoscopic instrument of claim 1, wherein the retractable tool actuator includes at least one of a motor driven by electrical power or a piezoelectric element driven by electrical power.

8. The endoscopic instrument of claim 1, wherein the retractable tool actuator is coupled to one or more electrical wires to receive electrical power and deliver the electrical power into the retractable tool to enable the retractable tool to perform electrocautery.

9. The endoscopic instrument of claim 1, wherein the retractable tool is attached to at least one of the inner cannula or the outer cannula.

10. The endoscopic instrument of claim 1, wherein the retractable tool actuator includes a control wire extending from the proximal end to the retractable tool, the control wire coupled to a biasing element that biases a position of the retractable tool relative to the tool channel.

11. The endoscopic instrument of claim 1, comprising a slot defined by the tool channel in which the retractable tool moves and that receives the retractable tool.

12. The endoscopic instrument of claim 1, comprising a plurality of electromagnets configured to selectively generate electromagnetic fields based on received electric currents to vary a magnetic force applied to a permanent magnet of the retractable tool to drive the retractable tool into or out of the channel.

13. The endoscopic instrument of claim 1, wherein the inner cannula rotates relative to the outer cannula.

14. A method of operating an endoscopic instrument, comprising:
positioning the endoscopic instrument in proximity to a site of a subject, the endoscopic instrument including an outer cannula and inner cannula disposed within the outer cannula, a tool channel defined within a radial wall of the outer cannula or positioned adjacent to the radial wall of the outer cannula;
receiving a control signal at a retractable tool actuator of the endoscopic instrument;
moving, by the retractable tool actuator responsive to the control signal, a retractable tool along the tool channel from a first position in which a distal tip of the retractable tool is within the tool channel to a second position in which a distal tip of the retractable tool extends beyond a distal end of the outer cannula; and
retrieving a sample of the subject from the site of the subject.

15. The method of claim 14, comprising:
delivering electrical power via electrical wires coupled to the retractable tool actuator to the retractable tool to perform electrocautery using the retractable tool.

16. The method of claim 14, comprising:
manipulating, by the retractable tool actuator, at least one of a frequency of movement of the retractable tool or a distance the retractable tool extends beyond the distal end of the outer cannula based on the control signal.

17. The method of claim 14, comprising:
operating at least one of a motor of the retractable tool actuator or a piezoelectric element of the retractable tool actuator to move the retractable tool from the first position to the second position.

18. The method of claim 14, comprising:
selectively generating, by a plurality of electromagnets of the endoscopic instrument, electromagnetic fields using received electric currents to vary a magnetic force applied to a permanent magnet of the retractable tool to move the retractable tool from the first position to the second position.

19. The method of claim 14, comprising moving the retractable tool within a slot defined by the tool channel.

20. The method of claim 14, comprising rotating the inner cannula relative to the outer cannula.
